(19) United States
(12) Patent Application Publication
(10) Pub. No.: US 2025/0144159 A1
(43) Pub. Date: May 8, 2025

(54) COMPOSITION COMPRISING A MIXTURE OF DHA AND/OR EPA AND A PHOSPHOLIPID OF PLANT ORIGIN

(71) Applicant: POLARIS, Quimper (FR)

(72) Inventors: Gildas BRETON, Quimper (FR); Manon LAOT, Quimper (FR); Lionel LARVOL, Quimper (FR)

(73) Assignee: POLARIS, Quimper (FR)

(21) Appl. No.: 18/837,082

(22) PCT Filed: Feb. 7, 2023
(86) PCT No.: PCT/EP2023/052967

§ 371 Date: Aug. 8, 2024

(30) Foreign Application Priority Data
Feb. 9, 2022 (FR) FR2201122

(51) Int. Cl. A61K 36/06; A61K 31/122; A61K 31/202; A61K 31/685; A61K 36/28; A61K 36/48; A61K 36/889

(52) U.S. Cl. CPC A61K 36/06; A61K 31/122; A61K 31/202; A61K 31/685; A61K 36/28; A61K 36/48; A61K 36/889

(57) ABSTRACT

A composition comprising a mixture of DHA and/or EPA in the form of glyceride or ethylester derived from at least one microorganism, such as a microalga, and at least one plant-derived phospholipid, in which the content of DHA and/or EPA in triglyceride form is between 10% and 90% by weight relative to the total weight of the composition, and the content of at least one plant-derived phospholipid, such as phosphatidylcholine, is between 10% and 90% by weight relative to the weight of composition. The method for manufacturing same and to the use thereof, in particular in the treatment of pathologies involving a deficiency of DHA and/or EPA, such as age-related macular degeneration or Alzheimer's disease.

The invention relates to a composition comprising a mixture of DHA (docosahexaenoic acid) and/or EPA (eicosapentaenoic acid) in the form of glycerides or ethyl ester derived from at least microorganism, such as a microalga, and at least one plant-derived phospholipid, the method for preparing same and use thereof as medication, for example in treating age-related macular degeneration, contributing DHA and/or EPA in the context of Alzheimer's disease and more generally in treating pathologies involving a DHA and/or EPA deficiency.

DHA is a fatty acid that is essential in the nervous tissues and in particular the brain and retina. The biosynthesis of DHA from precursors is very limited. An exogenous addition of DHA is then necessary (and hence this concept of indispensability) in particular during its formation in the embryo and the neonate but also in the case of neurodegenerative diseases.

Because of the aging of the population and the increase in the incidence of neurodegenerative diseases, developing DHA-based nutritional approaches for in particular mental-health prevention is a great challenge.

It is now well accepted that the favoured form of DHA intake in the brain via the haematoencephalic barrier (HEB) is lysophosphatidylcholine (lysoPC) where DHA is in Sn-2 position (Hachem et al, vol 53, Mol Neurbiol. 2016). In addition, a specific carrier (Mfsd2a) of lysoPC in the HEB has been identified, allowing preferential incorporation of DHA in the brain.

It has recently been demonstrated (Hachem et al, 2020, vol 12, Nutrients 2020, Bernoud-Hubac N. et al, vol 24, OCL 2017), that oral intake of lysoPC-DHA (or of AceDoPC, the molecular form mimicking lysoPC-DHA) improves the cerebral capture of DHA compared with an intake of DHA in triglyceride (TG) form.

It is accepted that plasmatic bioavailability of EPA and DHA is better when the latter are contributed in the form of krill phospholipids compared with the triglyceride form (Maki et al, 2009, Nutrition Research, vol 29, 2009). However, a supplementation of EPA and DHA on the triglyceride diet by a third or more compared with the phosphatidylcholine (PC) diet makes it possible to establish a similar plasmatic level of EPA and DHA (Ulven et al, 2010, vol 46, Lipids 2011). In other words, the phospholipid form improves the plasmatic bioavailability of EPA and DHA compared with the triglyceride form, but a supplementary intake (increased by one third) makes it possible to compensate for this difference.

In Wu et al, Lipids in Health and Disease, 2017), it was described that brain accretion of DHA in mice deprived of DHA before weaning is re-established after intake of DHA-PC or of DHA-TG and PC, at a level very slightly lower in the latter case (16.5% as against 14.72% in gas chromatography area (GC area)). DHA and glycerylphosphorylcholine (GPC) give a level comparable to DHA-PC: a separate intake of DHA and of PC (or of GPC) is a solution for contributing DHA to the brain.

At the hepatic level, the level of DHA is very quickly regained (two days after weaning) whether with DHA-PC or DHA-TG and PC or DHA-TG and GPC, for hepatic TGs and phospholipids (PLs)—Wu et al, Lipids in Health and Disease, 2017.

Surprisingly, at 4 days after weaning, the hepatic DHA level is higher for TG-DHA and PC or TG-DHA and GPC diets whether with regard to PLs (increase of 3 to 4 points %; GC area) or TGs (2.5% as against 3.5%)—Wu et al, Lipids in Health and Disease, 2017.

The erythrocytary accretion of DHA (which is said to reflect brain accretion of DHA), at 14 days after weaning, is greater for the PC-DHA diet (9.7% DHA; GC area) than for the TG-DHA and PC or TG-DHA and GPC diets (8.3%). The interpretation is, without being bound by this theory, perhaps passage in lysoPC-DHA form associated with albumin after hydrolysis with phospholipase A2 (or direct passage of PC-DHA)—Wu et al, Lipids in Health and Disease, 2017.

Other studies have shown only a slight difference in tissue accretion of DHA depending on the forms of intake. Unlike neonates or aged persons or in the case of diseases related to the nervous system, there is very little brain or ophthalmic accretion of DHA in adults with very little difference depending on the forms of intake. Thus it is difficult to observe significant differences in DHA accretion in target organs in healthy adults.

The sources of PL-DHA or PC-DHA are relatively rare. These have a marine origin and are found in particular in krill and fish roe (herring, cod). Lipids extracted from krill (zooplankton) have the particularity of being rich in PC (approximately 40%), but these PCs are mainly grafted with EPA. EPA represents approximately 28% of the PC fatty acids whereas DHA represents only approximately 14% thereof. DHA is preferentially in position Sn-2 of PC, EPA in position Sn1 and in position Sn-2.

Thus such sources of DHA and/or of EPA limit the quantities of products that it is possible to obtain, or would give rise to a complex and therefore expensive industrial production of products.

In addition, such sources may quite simply be prohibited for some people with sensitivities in terms of allergy to certain marine fauna, or quite simply having made a choice as to their dietary habits, as is the case with veganism. Thus, even if large- scale industrial production were possible for obtaining a product contributing DHA and/or EPA from krill (certainly involving high research and development costs with a process that was probably ultimately uneconomical/unecological), from fish or from fish roe, the market would be limited by the very nature of the sources of such products.

Thus there exists in the art a deficiency in obtaining a product contributing DHA and/or EPA that is reliable, easy of access, capable of large-scale industrial production and is acceptable to the greatest number of people. Furthermore, a means for obtaining a form as close as possible to lysoPC-DHA could have advantages in terms of efficacy in administering the product.

The object of the present invention therefore seeks to solve these deficiencies.

### SUMMARY

The object of the present invention relates to a composition comprising a mixture of DHA and/or EPA in glyceride or ethyl ester form derived from at least one microorganism, such as a microalga, and at least one plant-derived phospholipid, characterised in that:
the proportion of DHA and/or DPA in glyceride form, such as a triglyceride, or of ethyl ester or esters is between 10% and 90% by mass with respect to the total weight of the composition, and
the proportion of at least one plant-derived phospholipid, such as phosphatidylcholine, is between 10% and 90% by mass with respect to the mass of composition.

The object of the present invention thus relates to a method for manufacturing a composition according to the present invention comprising the following successive steps:

(a) obtaining an oil enriched with DHA and/or EPA in glyceride form, such as a triglyceride;

(b) adding at least one plant-derived phospholipid.

The step of extracting the oil from a microorganism, such as a microalga, is performed by heating under vacuum a suspension of microorganisms, such as microalgae, preferably ground, for example at 45-50° C., or even up to 60° C. Thus, at the end of fermentation, the cell walls are lysed by means of enzyme (advantageously a protease) to release the fat, which is then recovered by centrifugation.

The object of the present invention also relates to a composition according to the present invention for use thereof as a medication, preferentially as food supplement.

The object of the present invention relates more particularly to a composition as described herein for use thereof in treating pathologies involving a DHA and/or EPA deficiency, such as age-related macular degeneration, psychiatric disorders such as Alzheimer's disease, or more generally for psychiatric disorders, in particular pathologies related to one or more EPA-related disorders.

The object of the present invention relates more particularly to a composition as described herein for use thereof as a form of intake of DHA for developing the brain, in particular the brain of a human being, preferentially as from the foetus stage and a subsequent stage, such as the neonatal stage.

Definitions “DHA and/or EPA in glyceride form” means in the context of the present invention glycerol esters with docosahexaenoic acid (DHA) and/or eicosapentaenoic acid (EPA). The glycerides may be monoglycerides, diglycerides or triglycerides of DHA and/or EPA.

“DHA and/or EPA in the form of ethyl ester(s)” means, in the context of the present invention, an ethanol molecule bonded to DHA and/or EPA via an ester bond, i.e. DHA and/or EPA with a —COOEt group.

“Derived from at least one microorganism, such as a microalga” means, in the context of the present invention, that the origin of the DHA and/or of the EPA in glyceride form, such as a triglyceride, comes from at least one microorganism, such as a microalga. In other words, the origin of the DHA and/or EPA in glyceride form, such as a triglyceride, is traceable in the various steps of the methods for obtaining the composition according to the present invention.

“At least one plant-derived phospholipid” means, in the context of the present invention, that said at least one phospholipid comes from at least one plant. In other words, the origin of the phospholipid is traceable in the various steps of the methods for obtaining the composition according to the present invention.

“Oil enriched with DHA and/or EPA” means, in the context of the present invention, an oil having an EPA or DHA content greater than or equal to 300 mg/g, such as lying between 350 and 800 mg/g, for example 400 mg/L or 700 mg/g.

“Pathologies involving a DHA and/or EPA deficiency” means, in the context of the present invention, pathologies relating for example to a cardiovascular risk (thrombosis, excessively high level of triglycerides and/or of cholesterol in the blood, low blood pressure), to ocular fragility (related in particular to the retina), or to neurological pathologies (such as depression, neurodegenerative diseases such as Alzheimer's disease or epilepsy).

“Brain development” means, in the context of the present invention, neural development, or in other words the process of morphological and functional maturing of the encephalus.

### DETAILED DESCRIPTION

Composition:

The present invention therefore relates to the composition described above, the method for obtaining same and uses thereof as described above.

Preferably, the composition can furthermore be characterised in that:
said at least one microorganism, such as a microalga, is selected from the list consisting of a Traustochytride, such as Schizochytrium sp or Crythecodinium cohnii, Nannochloropsis, Isochrysis, Phaeodactylum or Nitzchia. There exist strains of microalgae, such as for example Crypthecodinium cohnii, Nannochloropsis, Isochrysis, Phaeodactylum or Nitzchia, which can produce PLs with a majority of PC, with even PC di-DHA;
said at least one plant-derived phospholipid is selected from the list consisting of sunflower oil, canola soil, soya oil and mixtures thereof;
preferably, said at least one plant-derived phospholipid is a plant extract selected from the list consisting of sunflower oil and soya oil;
preferably, the plant-derived phospholipid is rich in lecithin and/or phophatidylcholine. This is because phosphatidylcholine appears to have an effect on the haemato-encephalic barrier: it facilitates the passage of active principles;
the composition furthermore comprises DPAn-6;
the DPAn-6 content is between 0.1% and 20% by mass with respect to the total weight of composition;
the sum of DHA proportions in glyceride(s) form, such as a triglyceride, derived from at least one microorganism, such as a microalga, and the plant-derived phospholipid(s) portion is greater than or equal to 50% by mass with respect to the total weight of composition;
the EPA proportion is between 10% and 90% by mass with respect to the total weight of composition;
the DHA: EPA proportion is between 1:100 and 100:1 by mass, respectively;
said composition furthermore comprises at least one antioxidant the proportion of which is between 0.001% and 2% by mass with respect to the total weight of composition;
the antioxidant is selected from the following list: carotenoids, astaxanthin, zeaxanthin and lutein, preferentially astaxanthin;
the composition comprises at least one triglyceride with MCT short-chain fatty acids with a proportion preferentially between 1% and 50% by mass, such as 20% plus or minus 5% by mass, and/or
the composition is a food composition, referentially vegan.

More specifically, the composition according to the present invention can have the following characteristics.

In the current context of the strong trend towards vegan diets, the composition according to the present invention fits optimally for an intake of DHA and/or EPA of microalgal origin and of phosphatidylcholine (PC) (i.e. also an intake of choline) favouring brain accretion of DHA.

Thus the object of the present invention relates to a composition as described, characterised herein in that it is a vegan composition, i.e. it does not have any material directly of animal origin, and preferentially eco-friendly.

Thus the microalga oils containing DHA, for example based on Schizochytrium, or the EPA fraction after fractioning of the EPA and DHA oil that contains a little DHA, are characterised by the presence of DPA n-6. DPA n-6 is not present in fish oils (or very slightly <0.3% fatty acids), characteristic of microalga oils (for example, base oils with 400 mg of DHA/g of oil contain approximately 80 g of DPA n-6/g of oil).

In a particular embodiment, the composition (which can be vegan) according to the present invention comprises DPAn-6 in a proportion of between 0.5% and 19% by mass with respect to the total weight of composition, such as between 1% and 18%, between 5% and 17%, between 10% and 16% by mass with respect to the total weight of composition. More preferably, the composition (which can be vegan) according to the present invention comprises DPAn-6 in a proportion of 15% plus or minus 3% by mass with respect to the total weight of composition.

Even more preferably, the composition (which can be vegan) according to the present invention comprises DPAn-6 in a proportion of 10% plus or minus 3% by mass with respect to the total weight of composition.

It should be noted that, in the case of phospholipids with a lower PC titer, it will then be necessary to add more to achieve the above quantities and therefore the quantity of DHA will be less. Thus, for oils rich in EPA, there is only very little DPA n-6.

Some microalga oils, such as Nannochloropsis (photoautotrophic) or Schizochytrium (heterotrophic) (after fractioning of the EPA), do not contain eicosenoic acid C20:1 (C20:1 W7 eicosenoic and C20:1 W9 gadoleic) and docosenoic acid C22:1 (C22:1 W9 erucic and C22:1 W11 cetoleic). This is because C20:1 and C22:1 are more specific to zooplankton and copepods in particular (via the food chain, C20:1 and C22:1 are found in fish).

Thus, in a particular embodiment, the composition (which can be vegan) according to the present invention comprises:
eicosenoic acid C20:1 in a proportion of less than or equal to 5%, preferably less than or equal to 1%, with respect to the total weight of composition; and/or
docosenoic acid C22:1 in a proportion of less than or equal to 5%, preferentially less than or equal to 1%, with respect to the total weight of composition.

Preferably, the composition (which can be vegan) according to the present invention is:
free from eicosenoic acid C20:1; and/or
free from docosenoic acid C22:1.

In a particular embodiment, in the composition (which can be vegan) according to the invention, the sum of the DHA contents in glyceride(s) form, such as a triglyceride, derived from at least one microorganism, such as a microalga, and the plant-derived phospholipid(s) content is greater than or equal to 55% by mass with respect to the total weight of composition, preferentially greater than or equal to 60%, greater than or equal to 65%, greater than or equal to 70%, greater than or equal to 75%, greater than or equal to 80%, greater than or equal to 85%, greater than or equal to 90%, greater than or equal to 95%, or greater than or equal to 98%.

According to a particular embodiment, the EPA content included in the composition (which can be vegan) according to the present invention is between 10 and 70%, preferentially between 20% and 60% by mass with respect to the total weight of composition, for example between 30% and 50%, or between 35% and 40%, such as 37.5% plus or minus 2% by mass with respect to the total weight of composition.

In a particular embodiment, the DHA content included in the composition (which can be vegan) according to the present invention is between 20% and 80% by mass with respect to the total weight of composition, for example between 30% and 70%, or between 40% and 60%, such as 50% plus or minus 5% by mass with respect to the total weight of composition.

In a particular embodiment, the composition (which can be vegan) according to the present invention comprises a DHA: EPA proportion of between 1:9 and 75:1 by mass, respectively, preferentially between 1:8 and 50:1, between 1:7 and 25:1, between 1:6 and 10:1, between 1:5 and 5:1, between 1:4 and 4:1, between 1:3 and 3:1, between 1:2 and 2:1 or between 1.5:1 and 1.5:1 by mass.

In a particular embodiment, the composition (which can be vegan) according to the present invention furthermore comprises at least one antioxidant the proportion of which is between 0.003% and 1.5% by mass, preferentially between 0.005% and 1.4% by mass, between 0.01% and 1.3%, between 0.02% and 1.3%, between 0.05% and 1.2%, between 0.1% and 1.1%, between 0.5% and 1.0%, between 0.7% and 0.9%, such as 0.8% plus or minus 0.05% by mass with respect to the total weight of the composition.

In a particular embodiment, the object of the present invention relates to a composition (which can be vegan) that comprises between 10% and 90% by mass DHA coming from a microorganism, such as a microalga, and between 10% and 80% by mass phosphatidylcholine.

In a particular embodiment, the object of the present invention relates to a composition (which can be vegan) that comprises DPAn-6 in a proportion of between 1% and 19% by mass, preferentially between 5% and 18%, between 10% and 17%, between 12% and 16% or 15% plus or minus 0.5% by mass with respect to the total weight of composition.

Preferentially, the object of the present invention relates to a composition (which can be vegan) comprising between 20% and 80% by mass, for example between 30% and 70%, or between 40% and 60%, such as 50% plus or minus 5% by mass, DHA derived from a microorganism, such as a microalga, and between 20% and 70% by mass, for example between 30% and 60%, or between 40% and 50%, such as 40% plus or minus 5% by mass, phosphatidylcholine.

In a particular embodiment, the object of the present invention relates to a composition (which can be vegan), comprising between 10% and 90% by mass EPA derived from a microorganism, such as a microalga, and between 10% and 80% by mass phosphatidylcholine.

Preferentially, the object of the present invention relates to a composition (which can be vegan) comprising between 20% and 80% by mass, for example between 30% and 70%, or between 40% and 60%, such as 50% plus or minus 5% by mass, EPA derived from a microorganism, such as a microalga, and between 20% and 70% by mass, for example between 30% and 60%, or between 40% and 50%, such as 40% plus or minus 5% by mass phosphatidylcholine.

In a particular embodiment, the object of the present invention relates to a composition (which can be vegan) of DHA and/or EPA derived from a microorganism, such as a microalga, comprising phosphatidylcholine and at least one antioxidant, such as astaxanthin, at least one carotenoid, zeaxanthin, lutein, vitamin E, for example tocopherol acetate or tocotrienol; vitamin C or one of the analogues thereof, or natural polyphenols.

In a particular embodiment, the object of the present invention relates to a composition (which can be vegan) of DHA and/or of EPA coming from a microorganism, such as a microalga, with a proportion of chlorophyll of less than or equal to 5% by mass, preferentially less than or equal to 4%, less than or equal to 3%, less than or equal to 2%, or less than or equal to 1%. More preferentially, the vegan composition of DHA and/or of EPA coming from a microorganism, such as a microalga, is free from chlorophyll.

In a particular embodiment, the object of the present invention relates to a composition (which can be vegan) comprising DHA and/or EPA coming from a microorganism, such as a microalga, and phosphatidylcholine, with a proportion of chlorophyll of less than or equal to 5% by mass, preferentially less than or equal to 4%, less than or equal to 3%, less than or equal to 2%, or less than or equal to 1%. More preferentially, the vegan composition of DHA and/or of EPA coming from a microorganism, such as a microalga, and phosphatidylcholine is free from chlorophyll.

In a particular embodiment, the object of the present invention relates to a composition (which can be vegan) of DHA and/or EPA coming from a microorganism, such as a microalga, with a proportion of glycolipids of less than or equal to 5% by mass, preferentially less than or equal to 4%, less than or equal to 3%, less than or equal to 2%, or less than or equal to 1%. More preferentially, the vegan composition of DHA and/or of EPA coming from a microorganism, such as a microalga, is free from glycolipids

In a particular embodiment, the object of the present invention relates to a composition (which can be vegan) comprising DHA and/or EPA coming from a microorganism, such as a microalga, and phosphatidylcholine, with a proportion of glycolipids of less than or equal to 5% by mass, preferentially less than or equal to 4%, less than or equal to 3%, less than or equal to 2%, or less than or equal to 1%. More preferentially, the vegan composition comprising DHA and/or of EPA coming from an alga or a microalga and phosphatidylcholine is free from glycolipids. This is because glycolipids do not come from microalga oil resulting from culture thereof in heterotrophic mode.

In a particular embodiment, the object of the present invention relates to a composition (which can be vegan) of DHA and/or EPA derived from a microorganism, such as a microalga, and at least one triglyceride with short-chain fatty acids (MCTs), i.e. C1-C10, preferentially C2-C8, or C4-C6.

In a particular embodiment, the object of the present invention relates to a composition (which can be vegan) comprising DHA and/or EPA derived from a microorganism, such as a microalga, of phosphatidylcholine and at least one triglyceride with short-chain fatty acids (MCTs), i.e. C1-C10, preferentially C2-C8, or C4-C6.

This is because triglycerides with short-chain fatty acids (MCTs) can have a ketogenic action, which is an advantage in certain brain pathologies. Short-chain fatty acids can thus become the source of brain energy.

By way of illustration, in the case of Alzheimer's disease, the use of glucose (supplied via the digestive system) is reduced in the brain. MCTs then act as an energy source for the brain. Thus, in certain pathologies such as Alzheimer's disease or at extreme ages of life, it appears that the energy intake of the brain is disturbed or ineffective. In the case of Alzheimer's disease, the reduction in uptake of glucose by the brain for energy is impaired. This reduction appears before the cognitive decline syndromes and before the reduction in neuronal functions. Beta amyloid (the presence of an aggregate with Alzheimer's disease) has shown inhibiting activities on pyruvate dehydrogenase, thus limiting the production of acetyl-CoA, the fuel for the Krebs cycle for producing energy. The ketogenetic method for combating the deficit in glucose energy intake assumes great importance. Medium or short-chain fatty acids are ketogenetic. They are degraded by the mitochondria of the liver into ketone bodies (acetoacetate, beta-hydroxybutyrate) which, contributed to the brain by the blood, will be used as an alternative fuel by regenerating the acetyl COA then available for the Krebs cycle and the production of energy.

Adding these medium chain fatty acids (MCFAs) to the formulation is a real advantage. This addition can be done in their fatty acid form or in triglyceride form (such as MCTs). These MCFAs in addition have the particularity of being quickly absorbed and transported to the liver compared with longer fatty acids, which require activation via the carnitine system. Capric acids and more particularly caprylic acids are preferred.

Preferably, the object of the present invention relates to a composition (which can be vegan) comprising DHA and/or EPA derived from a microorganism, such as a microalga, and at least one triglyceride with short chain fatty acids (MCTs) in a proportion of between 1% and 50% by mass, for example between 5% and 40%, or between 10% and 30%, such as 20% plus or minus 5% by mass.

Preferably, the object of the present invention relates to a composition (which can be vegan) comprising DHA and/or EPA derived from a microorganism, such as a microalga, phosphatidylcholine and at least one triglyceride with short chain fatty acids (MCTs) in a proportion of between 1% and 50% by mass, for example between 5% and 40%, or between 10% and 30%, such as 20% plus or minus 5% by mass.

In a particular embodiment, the object of the present invention relates to a composition (which can be vegan) comprising DHA and/or EPA derived from a microorganism, such as a microalga, characterised in that this composition is adapted to allow an increase in bioavailability. Thus, preferably, the composition according to the invention is an emulsion, such as an emulsion, a microemulsion or a nanoemulsion, which can be a (micro-/nano-) oil-in-water or water-in-oil emulsion.

Preferably, the composition according to the invention is an autoemulsifiable composition.

Preferably, the composition according to the invention is an oil-in-water emulsion.

Preferably, the object of the present invention relates to a composition (which can be vegan) comprising DHA and/or EPA derived from a microorganism, such as a microalga, characterised in that this composition is adapted for autoemulsification, i.e. the composition emulsifies without external mechanical input.

In a particular embodiment, the object of the present invention relates to a composition (which can be vegan) comprising DHA and/or EPA derived from a microorganism, such as a microalga, and phosphatidylcholine, characterised in that this composition is adapted to allow an increase in bioavailability. Thus, preferably, the composition according to the invention is an emulsion, such as an emulsion, a microemulsion or a nanoemulsion, which may be a (micro-/nano-) oil-in-water or water-in-oil emulsion.

Preferably, the object of the present invention relates to a composition (which can be vegan) comprising DHA and/or EPA derived from a microorganism, such as a microalga, and phosphatidylcholine, characterised in that this composition is adapted for autoemulsification.

In a particular embodiment, the object of the present invention relates to a food composition (which can be vegan), such as a food supplement that can take the form of a solid or a liquid.

Preferably, the food composition (which can be vegan) according to the present invention can be in pre-dosed form.

Preferably, the food composition (which can be vegan) according to the invention can take the form of a capsule, a tablet, an encapsulated liquid or a liquid contained in an ampoule.
Methods for Obtaining

Oils enriched with DHA and/or EPA may be commercial (marketed for example under the trade name Omegavie Algae Oils® from the company Polaris).

Nevertheless, the following three procedures (hereinafter referred to as “options”) make it possible to obtain oils enriched with DHA and/or EPA.

The first option (as illustrated in example 1 below) consists of a method comprising the following steps:
(a1) a step of reaction between a microorganism oil comprising omega-3 polyunsaturated fatty acids in triglyceride form and an alcohol in the presence of a chemical or enzymatic catalyst,
(b1) a step of concentrating with omega-3 polyunsaturated fatty acids by molecular distillation under high vacuum in a wiped-film evaporator coupled to a rectification column comprising at least seven theoretical plates, thus making it possible to obtain DHA in ethyl ester (EE) form; and
(c1) a step of forming glycerides of DHA comprising the additional steps (d1) and (e1):
(d1) a step of restructuring monoglycerides, diglycerides and triglycerides of omega-3 polyunsaturated fatty acids in the presence of enzyme and glycerol; and
(e1) a step of short-path molecular distillation under vacuum.

An illustration of this first option is given in example 1.1 below.

The second option (as illustrated in example 2 below) consists of a method for enriching with DHA or EPA starting from the same source as option 1. In particular the method according to option 2 is a method for fractioning fatty acids with two difference carbons by molecular distillation (including fractionation between the EPA and DHA), characterised in that it comprises the following steps:

(a2) a step of reaction between a microorganism oil comprising omega-3 polyunsaturated fatty acids in triglyceride form and an alcohol in the presence of a chemical or enzymatic catalyst;

(b2) a (e.g. first) step of molecular distillation under high vacuum of the oil derived from step (a2), in a wiped-film evaporator coupled to a rectification column comprising at least seven theoretical plates, and recovery of a first residue and of a (first) distillate;

(c2) a subsequent (e.g. second) step of molecular distillation under high vacuum of the residue recovered at step (b2), in said wiped-film evaporator coupled to the rectification column comprising at least seven theoretical plates, and recovery of a second residue and of a (second) distillate;

(d2) an optional step of restructuring monoglycerides, diglycerides and triglycerides of omega-3 polyunsaturated fatty acids in the presence of enzyme and glycerol; and

(e2) a step of short-path molecular distillation under vacuum.

The two steps (b2) and (c2) make it possible to separate the DHA and the EPA. With a single step this does not appear to be possible. The equipment (7 plates) appears to be decisive.

At step (c2), the DHA and EPA are in the form of ethyl esters. It is thus possible to restore them to the form of diglycerides.

An illustration of this second option is given in example 1.2 below.

The third option (as illustrated in example 3 below) consists of a method for enriching an oil with EPA comprising the following steps:

(a3) a step of reaction between a microorganism oil comprising EPA and an alcohol in the presence of a chemical catalyst;

(b3) a step of concentration and purification of fatty-acid ethyl esters including ethyl esters of EPA by short-path molecular distillation under high vacuum;

(c3) a step of concentration with eicosapentaenoic acid by molecular distillation on rectification column;

(d3) a step of restructuring monoglycerides, diglycerides and triglycerides of omega-3 polyunsaturated fatty acids in the presence of enzyme and glycerol; and

(e3) a step of short-path molecular distillation under vacuum.

The starting oil at step (a3) can for example be a microalga oil of the genera Nannochloropsis, Isochrysis, Phaeodactylum or Nitzchia. An illustration of this third option is given in example 1.3 below.

At step (e3) of the method, the EPA in the form of ethyl esters (EEs) is obtained. It is possible to put it in the form of glycerides by adding the following steps:

(f3) a step of restructuring monoglycerides, diglycerides and triglycerides of omega-3 polyunsaturated fatty acids in the presence of enzyme and glycerol; and

(g3) a step of short-path molecular distillation under vacuum.

Another aspect of the present invention relates to a method for manufacturing a composition as described above. This method can comprise the following successive steps:

(a) obtaining an oil enriched in DHA and/or EPA in glyceride form, such as a triglyceride, for example by any one of the methods cited above;

(b) adding at least one phospholipid of plant origin.

Any addition technique applicable in this case can be used. Preferably, the oils of DHA and/or EPA and of at least one plant-derived phospholipid are mixed at high speed and/or for sufficient time to obtain sufficient homogeneity of the mixture. In particular, gradual putting under vacuum while heating under stirring to complete dissolution of the phospholipid in the mixture can be implemented.

In particular, the oils of DHA and/or EPA and of at least one plant-derived phospholipid are mixed under vacuum in order to avoid oxidation thereof or more generally the incorporation of gas in the mixture.

Preferably, the method comprises a step wherein the mixture of oils of DHA and/or EPA and of at least one plant-derived phospholipid is heated at atmospheric pressure, for example after having been mixed under vacuum, at a temperature of between 5° C. and 60° C., preferentially between 45° C. and 50° C.

Advantageously, the method comprises a step wherein the mixture of oils of DHA and/or EPA and of at least one plant-derived phospholipid is heated at atmospheric pressure under inert gas, such as nitrogen, or preferentially under vacuum.
Applications

The object of the present invention thus relates to a composition as described herein for use thereof in treating pathologies involving a DHA and/or EPA deficiency.

In particular, an intake of PC and of DHA will enable the organism to synthesise LysoPC-DHA, which is a favoured molecular form for carrying DHA to the brain in particular (PC is also a source of choline).

Preferably, the object of the present invention relates to a composition as described herein for use thereof in treating pathologies involving a DHA and/or EPA deficiency in a patient having an age greater than or equal to 40 years, greater than or equal to 45 years, greater than or equal to 50 years, greater than or equal to 55 years, greater than or equal to 60 years, greater than or equal to 65 years, greater than or equal to 70 years, greater than or equal to 75 years, greater than or equal to 80 years, greater than or equal to 85 years, greater than or equal to 90 years or greater than or equal to 95 years.

In a particular embodiment, the object of the present invention relates to a composition as described herein for use thereof in treating cardiovascular diseases.

Preferably, the object of the present invention relates to a composition as described herein for use thereof in treating cardiovascular diseases in a patient having an age greater than or equal to 40 years, greater than or equal to 45 years, greater than or equal to 50 years, greater than or equal to 55 years, greater than or equal to 60 years, greater than or equal to 65 years, greater than or equal to 70 years, greater than or equal to 75 years, greater than or equal to 80 years, greater than or equal to 85 years, greater than or equal to 90 years or greater than or equal to 95 years.

Preferably, the object of the present invention relates to a composition as described herein for use thereof in treating thrombosis.

Preferably, the object of the present invention relates to a composition as described herein for use thereof in treating thrombosis in a patient having an age greater than or equal to 40 years, greater than or equal to 45 years, greater than or equal to 50 years, greater than or equal to 55 years, greater than or equal to 60 years, greater than or equal to 65 years, greater than or equal to 70 years, greater than or equal to 75 years, greater than or equal to 80 years, greater than or equal to 85 years, greater than or equal to 90 years or greater than or equal to 95 years.

Preferably, the object of the present invention relates to a composition as described herein for use thereof in treating disorders related to an excessively high level of triglycerides and/or of cholesterol in the blood.

Preferably, the object of the present invention relates to a composition as described herein for use thereof in treating disorders related to an excessively high level of triglycerides and/or of cholesterol in the blood in a patient having an age greater than or equal to 40 years, greater than or equal to 45 years, greater than or equal to 50 years, greater than or equal to 55 years, greater than or equal to 60 years, greater than or equal to 65 years, greater than or equal to 70 years, greater than or equal to 75 years, greater than or equal to 80 years, greater than or equal to 85 years, greater than or equal to 90 years or greater than or equal to 95 years.

In a particular embodiment, the object of the present invention relates to a composition as described herein for use thereof in treating ocular weakness, in particular related to the retina.

In a particular embodiment, the object of the present invention relates to a composition as described herein for use thereof in treating macular degeneration, in particular age related. This is because an intake of PC-DHA is also important as from the youngest age and during gestation for forming the retina (visual acuity). Thus one of the objectives of the present invention can be an intake of PC-DHA in the mother and also in neonates by administering the composition according to the present invention.

Preferably, the object of the present invention relates to a composition as now described for use thereof in treating disorders related to ocular weakness, in particular relating to the retina, such as macular degeneration, in particular age related, in a patient having an age greater than or equal to 40 years, greater than or equal to 45 years, greater than or equal to 50 years, greater than or equal to 55 years, greater than or equal to 60 years, greater than or equal to 65 years, greater than or equal to 70 years, greater than or equal to 75 years, greater than or equal to 80 years, greater than or equal to 85 years, greater than or equal to 90 years or greater than or equal to 95 years.

In a particular embodiment, the object of the present invention relates to a composition as described herein for use thereof in treating neurological disorders, such as depression, neurodegenerative illness or epilepsy.

Preferably, the object of the present invention relates to a composition as described herein for use thereof in treating a neurodegenerative illness such as Alzheimer's disease, Parkinson's or sclerosis, such as amyotrophic sclerosis.

Preferably, the object of the present invention relates to a composition as described herein for use thereof in treating a neurodegenerative disease such as Alzheimer's disease, Parkinson's or sclerosis, such as amyotrophic sclerosis, in a patient having an age greater than or equal to 40 years, greater than or equal to 45 years, greater than or equal to 50 years, greater than or equal to 55 years, greater than or equal to 60 years, greater than or equal to 65 years, greater than or equal to 70 years, greater than or equal to 75 years, greater than or equal to 80 years, greater than or equal to 85 years, greater than or equal to 90 years or greater than or equal to 95 years.

In a particular embodiment, the object of the present invention relates to a composition as described herein for use thereof in treating a patient with an age greater than or equal to 40 years, greater than or equal to 45 years, greater than or equal to 50 years, greater than or equal to 55 years, greater than or equal to 60 years, greater than or equal to 65 years, greater than or equal to 70 years, greater than or equal to 75 years, greater than or equal to 80 years, greater than or equal to 85 years, greater than or equal to 90 years or greater than or equal to 95 years.

Preferably, the object of the present invention relates to a composition as described herein for use thereof in treating a patient with an age greater than 60 years having a pathology or a risk selected from cardiovascular diseases, thrombosis, disorders related to an excessively high level of triglycerides and/or of cholesterol in the blood, disorders related to ocular weakness, disorders related to the retina such as macular degeneration, neurological disorders, such as depression or epilepsy, or a neurodegenerative disease such as Alzheimer's disease, Parkinson's or sclerosis.

### EXAMPLES

Implementations of the present invention will be described below by way of non-limitative examples.

### Example 1: Method for Obtaining Microalga Oils

### Example 1.1.: Concentrating a Microalga Oil with DHA Mainly in Glyceride Form

This is a method for manufacturing an oil according to option 1 described above. This method is detailed in the patent document FR 3103355.

The method is implemented using a raw oil produced by the microalga strain Schizochytrium sp T18 marketed by the company Mara Renewables Corporation.

The oil initially contains 329 mg/g of DHA. The objective here is at a minimum to double the concentration of DHA.

### Step (i): Transesterification

A transesterification reaction is implemented on a biomass of 1800 kg of microalga oil using 450 kg of ethanol and 21.6 kg of sodium ethylate, in a suitable reactor.

The reaction temperature is 50° C. and the reaction time is 1 h. At the end of the reaction, the excess ethanol is evaporated under vacuum, then the mixture is cooled to a temperature of approximately 30° C. and then subjected to settling for 1 h. The light phase is recovered and then the glycerol is drained off. A second settling is implemented for 30 sec. The glycerol and the residual monoglycerides are drained off.

Washing with acidic water is next performed adding 17% demineralised water containing 2.5% phosphoric acid (75%) under stirring for 20 sec. The mixture is settled for 20 sec and the aqueous phase is drained off. Drying under vacuum follows (pressure<90 mbar) at 60° C. for a time greater than 2 h.

At the end of this step, the oil contains 329 m/g of DHA in the form of ethyl esters.

Step (ii): Increasing the DHA concentration by molecular distillation

The oil is next conveyed into a degasser and then passes through a wiped-film evaporator. The vapours are next distilled through a rectification column that is coupled to the evaporator, supplied by the company UIC GmbH. A reflux of the distillate, i.e. reintroduction of the distillate into the column, can make it possible to increase the separative efficacy of the column. The column used contains approximately seven theoretical plates. The distillation residue is recovered and represents the DHA-enriched fraction.

The operational conditions are as follows: T° of the evaporator: 225° C.; Vacuum of the rectification column: 0.1 mbar; Reflux ratio 75%, T° (column top): 195° C.

Table 1 below compares the fatty acid profile of the oil obtained after this step (ii) with the initial fatty acid profile before the first step (i). The quantities are expressed in mg of each fatty acid per g of oil.

TABLE 1

| | Fatty acid composition | Fatty acid composition |
|---|---|---|
| | before step (i) | after step (ii) |
| C4:0 | 1.867 | — |
| C6:0 | 1.808 | — |
| C12:0 | 7.542 | — |
| C14:0 | 89.582 | 4.428 |
| C15:0 | 2.478 | — |
| C16:0 | 153.76 | 10.206 |
| C16:1 | 69.696 | 5.611 |
| C18:0 | 6.133 | — |
| C18:1 n9 cis | 19.555 | 8.442 |
| C18:1 n7 cis | 89.271 | 17.745 |
| C18:2 n6 cis | 10.651 | 3.487 |
| C20:4 n3 | 3.055 | 5.572 |
| EPA | 11.294 | 18.856 |
| C22:5 n6 | 55.904 | 125.453 |
| DHA | 329.162 | 757.958 |

More than 97% of the DHA is preserved in the residue recovered. The quantity of DHA has changed from 329 mg/g of DHA in ethyl ester form to 758 mg/g.

Step (iii): Synthesis of triglycerides, diglycerides and monoglycerides

The reaction takes place in a reactor in the presence of immobilised enzyme. This is a lipase of fraction B of Candida antarctica (marketed under the name Lipozym 435 by the company Novozym).

480 g of distillation residue containing approximately 758 mg/g of DHA, 23 g of glycerol and 20 g of enzyme is introduced into the reactor.

The reaction takes place under moderate stirring, at a temperature controlled around 60° C., under vacuum with evaporation and condensation of the ethanol generated. The reaction time is 25 h.

The yield of the reaction is more than 95%.

At the end of this step, the oil has the following glyceride profile (Table 2):

TABLE 2

| Ethyl esters | Monoglycerides | Diglycerides | Triglycerides |
|---|---|---|---|
| 8.6% | 5.7% | 35.8% | 49.9% |

Step (iv): Short-path molecular distillation

After synthesis, the product is taken up to pass into a short-path distillator (marketed by the company UIC GmbH) to eliminate the residual ethyl esters and the odorous volatiles, but also to inactivate a potential residual enzymatic activity.

The conditions are a wall temperature of the evaporator of between 160 and 220° C. and a vacuum below 0.02 mbar. Preferably, the vacuum is 0.005 mbar to make it possible to reduce the distillation temperature. At the end of this step the oil has the profile indicated in Table 3:

TABLE 3

| Compounds | Units | Values (Example 1) |
|---|---|---|
| DHA | mg/g | 758 |
| Saturated fatty acids (SFA) | mg/g | 15 |
| Triglycerides (TG) | % | 63 |
| Diglycerides (DG) | % | 36 |
| Monoglycerides (MG) | % | 2 |
| Ethyl esters/free fatty acids | mg/g | 1.2 |
| (EE/FFA) | | |
| EPA | mg/g | 20 |
| ETA n-3 | mg/g | 6 |
| DPA n-6 | mg/g | 105 |

### Example 1.2: EPA/DHA Fractionation by Molecular Distillation and Obtaining of Microorganism Oil Compositions Enriched with Eicosapentaenoic Acid and Docosahexaenoic Acid

This is a method for manufacturing an oil according to option 2 described above. This method is detailed in the patent document FR 3108622.

The method according to the invention is implemented using a raw oil produced by the microalga strain Schizochytrium sp marketed by the company DSM under the trade name Life's DHA 60.

The profile of the oil is as follows:
360 mg/g of DHA, in mg/g of composition,
184 mg/g of EPA, in mg/g of composition,
94.1% triglycerides,
5.4% diglycerides,
0.4% monoglycerides.

The objective here is at a minimum to double the concentration of EPA and DHA.

### Step (i): Transesterification

A transesterification reaction is implemented on a biomass of 19 kg of microalga oil using 4.75 kg of ethanol and 222 g of sodium ethylate, in a suitable reactor.

The reaction temperature is 50° C. and the reaction time is 1 h. At the end of the reaction, the excess ethanol is evaporated under vacuum, then the mixture is cooled to a temperature of approximately 30° C. and then subjected to settling for 1 h. The light phase is recovered and then the glycerol is drained off. A second settling is implemented for 30 sec. The residual glycerol and monoglycerides are drained off.

Washing with acidic water is next implemented by adding 3.2 kg of demineralised water containing 76.4 g of phosphoric acid (75%) under stirring for 20 sec. The mixture is settled for 20 sec and the aqueous phase is drained off. Drying under vacuum follows (pressure<90 mbar at 60° C. for a time greater than 2 h.

At the end of this step, the oil contains 192 mg/g of EPA and 374 mg/g of DHA in the form of ethyl esters.

Step (ii): First molecular distillation step

The oil is next conveyed into a degasser and then passes through a wiped-film evaporator. The vapours are next distilled through a rectification column that is coupled to the evaporator supplied by the company UIC GmbH.

The aim is here to eliminate the lightest fatty acids while preserving the DHA and EPA. The column used contains seven theoretical plates. The distillation residue is recovered and represents the fraction enriched with EPA and DHA. The operational conditions are as follows: T° of the evaporator: 225° C.; Vacuum of the rectification column: <0.1 mbar; Reflux ratio 70%, T° (column bottom): 190° C., To (column top): 135° C.

At the end of this step, a residue fraction and a distillate fraction are obtained. The residue fraction contains 256 mg/g of EPA and 520 mg/g of DHA. The yield is 68%.

Step (iii): Second molecular distillation step

The operation of step (ii) is performed again on the residue. The latter is therefore conveyed again into the degasser and then passes through the wiped-film evaporator. The vapours are next distilled through the rectification column, coupled to the evaporator as in the case of the preceding step (ii). The aim is here to separate the DHA and EPA.

The operational conditions are as follows: T° of the evaporator: 235° C.; Vacuum of the rectification column: less than 0.05 mbar; Reflux ratio 60%, To (column bottom): 202° C., T° (column top): 160° C.

At the end of this step, a residue fraction and a distillate fraction are obtained. The distillate fraction contains 733 mg/g of EPA and 70 mg/g of DHA. The yield of this fraction is 84% EPA and 4% DHA. The residue fraction contains 706 mg/g of DHA and 83 mg/g of EPA. The yield of this fraction is 93% DHA and 22% EPA.

In Table 4 below, the detailed fatty acid profile of the distillate and residue fractions obtained at the end of step (iii) is indicated (in mg/g of composition).

TABLE 4

| | DISTILLATE | RESIDUE |
|---|---|---|
| C14:0 | 0 | 0 |
| C15:0 | 0 | 0 |
| C16:0 | 5.3 | 0 |
| C18:0 | 13.5 | 0 |
| C18:1 n9 cis | 27.6 | 0 |
| C18:2 n6 cis | 2.6 | 0 |
| C20:0 | 12.2 | 3.5 |
| C20:3 n6 | 2.1 | 0.3 |
| C21:0 | 5.0 | 0 |
| C20:1 n7 | 0.0 | 0.3 |
| C20:4 n6 | 54.9 | 4.4 |
| C20:3 n3 | 5.9 | 0 |
| UNKNOWN | 0.0 | 0 |
| UNKNOWN | 1.8 | 0.8 |
| C20:4 n3 | 22.4 | 3.7 |
| EPA | 733.4 | 83.2 |
| C22:0 | 0 | 2.6 |
| UNKNOWN | 5.0 | 5.2 |
| C22.4 n6 | 0.0 | 0 |
| C22:5 n6 | 4.4 | 30.7 |
| UNKNOWN | 0.0 | 1.8 |
| C22:5 n3 | 5.9 | 85.3 |
| DHA | 70.1 | 730 |

### Example 1.3: Obtaining Microalga Oil Comprising EPA

This is the method for manufacturing an oil according to option 3 described above.

The starting oil is a raw oil produced by the strain Nannochloropsis sp obtained after culture of the biomass for around a week under photoautotrophic condition. The fat is extracted from the biomass by ethanolic maceration. After separation of the remaining solid phase, the ethanol is evaporated under vacuum.

The EPA produced by photoautotrophy is incorporated in the membrane lipids, either in glycolipid form, or in phosolipid form, or in the form of free fatty acids. The triglyceride form is little represented.

### Step (i): Transesterification

A transesterification reaction is implemented on a biomass of 100 kg of microalga oil using approximately 200 kg of ethanol and 10 kg of sulfuric acid, in a suitable reactor (reflux 60° C.>10 h). The transesterification is implemented by acid method, with a sulfuric acid.

Step (ii): Concentrating EPA by short-path molecular distillation under vacuum

The conditions are a wall temperature of the evaporator of between 175 and 180° C. and a vacuum of less than 0.1 mbar.

Step (iii): Concentrating EPA by molecular distillation on rectification column

The operational conditions are as follows: T° of the evaporator: 230° C.; Vacuum of the rectification column: 0.07 mbar; Reflux ratio 65%, T° (column top): 107° C., T° (column bottom): 147° C. The quantity of EPA in the form of ethyl esters is 735 mg/g.

Examples of compositions according to the present invention

Plant-derived oils used:
soya oil 50% PC
sunflower oil 90% PC
non-concentrated Schizochytrium sp oil: 40% DHA
concentrated Schizochytrium sp oil: 70% DHA
Schizochytrium sp oil rich in EPA and DHA: 20% EPA and 30% DHA
palm oil with short-chain triglycerides (MCTs)
10% concentrated astaxanthin.
   Method for manufacturing a composition according to the present invention:
adding various elements in one and the same container
stirring and gradual putting under vacuum
heating the mixture under stirring until a homogeneous mixture is obtained
return to atmospheric pressure by adding inert gas.
   Table 5: Summary table of compositions obtained:

TABLE 5

| Compositions | Ingredients | Proportions (by mass) |
|---|---|---|
| A | Non-concentrated | 50% |
| | Schizochytrium s oil | |
| | according to the | |
| | manufacturing method XX | |
| | 50% PC soya oil | 50% |
| B | Concentrated | 49.9% |
| | Schizochytrium s oil | |
| | according to the | |
| | manufacturing method XX | |
| | 50% PC soya oil | 50% |
| | Astaxanthin | 0.1% |
| C | Non-concentrated | 20% |
| | Schizochytrium s oil | |
| | according to the | |
| | manufacturing method XX | |
| | Concentrated | 57% |
| | Schizochytrium s oil | |
| | according to the | |
| | manufacturing method XX | |
| | 90% PC sunflower oil | 22.9% |
| | Astaxanthin | 0.1% |
| D | Concentrated | 25% |
| | Schizochytrium s oil | |
| | according to the | |
| | manufacturing method XX | |
| | 50% PC soya oil | 60% |
| | Palm oil with short-chain | 15% |
| | triglycerides | |
| E | Schizochytrium sp oil rich | 17% |
| | in EPA and DHA: 20% | |
| | EPA and 30% DHA | |
| | Concentrated | 60% |
| | Schizochytrium s oil | |
| | according to the | |
| | manufacturing method XX | |
| | 90% PC sunflower oil | 19.9% |
| | Astaxanthin | 0.1% |

### Comparative example between a composition according to the present invention and a composition according to the prior art

TABLE 6
Composition C versus krill oil

| Compounds | Composition C | Krill oil |
|---|---|---|
| Phospholipid fraction | Min 30% by mass | 40% by mass |
| Phosphatidylcholine | Min 25% by mass | 30% by mass |
| EPA | Min 5 mg/g as triglycerides | Min 120 mg/g |
| DHA | Min 340 mg/g as | Min 55 mg/g |
| | triglycerides | |
| Total omega-3 | Min 345 mg/g | Min 220 mg/g |
| Astaxanthin | 10 mg/100 g | 10 mg/100 g |

It was found that the quantity of phosphatidylcholine in composition C is sufficient to produce emulsifying properties similar to krill oil.

Composition C has higher levels of DHA. The level of EPA can be increased if required. Thus the levels of DHA and EPA can be adapted according to the treatment sought.

The quantity of astaxanthin can also be modified according to the treatment sought.

It can nevertheless be found that composition C comprises 6 times more DHA and 1.5 times more omega-3 then krill oil.

### Example 3: In Vivo Tests in Rats: the Supposed Action Mechanism

Knowing that, during digestion, DHA is very quickly hydrolysed from PC (phosphatidylcholine) and that hepatic resynthesis of lysoPC-DHA in the presence of choline is active, the composition according to the present invention comprising PC and DHA appears to be a very well adapted form for contributing DHA to the target tissues.

It is well known that, after food intake, phospholipase A2 is very quickly active. The phospholipids ingested quickly undergo hydrolysis. DHA, being naturally in Sn-2, is released in the form of a free fatty acid. The latter is then available for intestinal absorption thereof. Several studies have shown better incorporation of DHA in erythrocytes when it is contributed in the form of PL-DHA followed by mono-DHA and then TG-DHA (erythrocytary accretion of DHA is considered to be a marker of cerebral accretion thereof). In this case, the TG or Mono were not supplemented with plant PLs.

Phospholipids are amphiphilic molecules conferring autoemulsification properties thereof, as what happens during the digestion of fatty acids. Emulsification is well recognised for improving the intestinal absorption of fatty acids in humans (Raatz S. K. et al, J of Am. Diet. Ass., vol. 109, 2009; Couëdelo L. et al, Food Funct. vol 6, 2015; Couëdelo L. et al. OCL, vol 24, 2017; Ottestad I. et al, J. of Nutr. Sci. vol 5, 2016). Thus an intake of lecithin improves the emulsification of lipids or fatty acids and the plasmatic bioavailability thereof.

A study with DHA oils in triglyceride form supplemented with plant phospholipids (PCs) compared with a regime of phosphatidylcholine-THA extracted from cod roe (composition of the company Arctic Nutrition AS of make Romega®) with an identical contribution of DHA showed, 10 h after intake, an equivalent plasmatic bioavailability of DHA (identical proportion of DHA). Regarding the proportions of DHA in the phospholipidic fraction of the plasma, a slightly greater proportion of DHA was noted with the PC-DHA regime (+approximately 15%). By analysing the distribution of DHA on the glycerol skeleton of the plasmatic phospholipids, it was observed that DHA is very mainly grafted in position Sn-2 (approximately 80% of the DHA) with a very slight advantage for the PC-DHA regime. With the TG-DHA and plant PL (PC) regime (without DHA), almost as much PC-DHA is therefore found as with the PC-DHA regime. This clearly proves that PC-DHA is hydrolysed by phospholipase A2 and that there is indeed resynthesis of PC-DHA Sn- 2 at the enterocytes before release into the plasma.

A better intake of DHA and/or EPA in the brain in a specific form was able to be determined here. Thus the best known form of contribution of DHA, or even EPA, to the brain is lysophosphatidylcholine (lysoPC) with DHA in position Sn2 and/or EPA in position Sn1 and in Sn2. The sources of DHA in lysoPC form are rare and expensive. It is a case of krill oils and fish roe oils. The present invention makes it possible to offer not only an alternative to these previous forms but offers a composition with technical effects that can be targeted according to the proportion and nature of the fatty acids that it contains (DHA and/or EPA).

1. A composition comprising a mixture of DHA and/or EPA in the form of glyceride(s) or ethyl ester(s) derived from at least one microorganism, such as a microalga, and at least one plant-derived phospholipid, wherein:
the proportion of DHA and/or EPA in glyceride form, such as a triglyceride, or of ethyl ester or esters, is between 10% and 90% by mass with respect to the total weight of the composition, and
the proportion of at least one plant-derived phospholipid, such as phosphatidylcholine, is between 10% and 90% by mass with respect to the mass of composition.

2. The composition according to claim 1, wherein said at least one microorganism is selected from the list consisting of a Traustochytride, such as Schizochytrium sp or Crythecodinium cohnii, Nannochloropsis, Isochrysis, Phaeodactylum or Nitzchia.

3. The composition according to claim 1, wherein said at least one plant-derived phospholipid is selected from the list consisting of sunflower oil, canola oil, soya oil and mixtures thereof.

4. The composition according to claim 1, wherein the composition furthermore comprises DPAn-6 and in that the proportion thereof is between 0.1% and 20% by mass with respect to the total weight of composition.

5. The composition according to claim 1, wherein the sum of the proportion of DHA in glyceride form, such as a triglyceride, derived from at least one microorganism and the proportion of plant-derived phospholipid(s) is greater than or equal to 50% by mass with respect to the total weight of the composition.

6. The composition according to claim 1, wherein the DHA: EPA proportion is between 1:100 and 100:1 by mass, respectively.

7. The composition according to claim 1, wherein said composition furthermore comprises at least one antioxidant, the proportion of which is between 0.001% and 2% by mass with respect to the total weight of composition and is selected from the following list: carotenoids, astaxanthin and lutein, preferentially astaxanthin.

8. The composition according to claim 1, wherein the composition comprises at least one triglyceride with short-chain fatty acids MCTs with a proportion of preferentially between 1% and 50% by mass, such as 20% plus or minus 5% by mass.

9. The composition according to claim 1, wherein the composition is a food composition, preferentially vegan.

10. The composition according to claim 1, for use thereof as a medication, preferentially as a food supplement.

11. The composition according to claim 1 for use thereof in treating pathologies involving a DHA and/or EPA deficiency, such as age-related macular degeneration, psychiatric disorders such as Alzheimer's disease, in particular pathologies related to one or more disorders related to EPA.

12. The composition according to claim 1 for use thereof as a form of intake of DHA for developing the brain.
